(19) United States
(12) Patent Application Publication
(10) Pub. No.: US 2026/0072005 A1
(43) Pub. Date: Mar. 12, 2026

(54) WATER QUALITY DETERMINATION DEVICE, WATER QUALITY DETERMINATION METHOD, AND FLOW CELL

(71) Applicant: WOTA CORP., Tokyo (JP)

(72) Inventors: Kyungju KIM, Tokyo (JP); Hodaka MUKOHARA, Tokyo (JP); Yoichi YOSHIDA, Tokyo (JP)

(21) Appl. No.: 19/231,355

(22) Filed: Jun. 6, 2025

(30) Foreign Application Priority Data
Sep. 6, 2024 (JP) 2024-153929

(51) Int. Cl. G01N 33/18; G01N 21/01; G01N 21/41

(52) U.S. Cl. CPC G01N 33/1893; G01N 21/41; G01N 2021/0193

(57) ABSTRACT

Provided is a water quality determination device and a water quality determination method capable of improving determination accuracy of water quality while reducing cost. The water quality determination device includes a first light emitting unit and a second light emitting unit that emit lights toward a flow path, a first light receiving unit that receives the light emitted from the first light emitting unit through the flow path, and a second light receiving unit that receives the light emitted from the second light emitting unit through the flow path. An optical path length from the second light emitting unit to the second light receiving unit is longer than an optical path length from the first light emitting unit to the first light receiving unit.

### CROSS-REFERENCE TO RELATED APPLICATION

The present application claims the benefit of Japanese Patent Application No. 2024-153929, filed on Sep. 6, 2024. The disclosures of the above-mentioned application are incorporated herein by reference in their entirety.

### FIELD OF THE INVENTION

The present invention relates to a water quality determination device, a water quality determination method, and a flow cell.

### BACKGROUND OF THE INVENTION

### [Regarding Water Quality Determination Device and Water Quality Determination Method]

Conventionally, a method for determining a water quality of water is known in which a turbidity of water is measured using a turbidity meter, and the water quality of the water is determined based on the measured turbidity of the water (Patent Document 1, etc.). Generally, the turbidity meter includes a light emitting unit that emits a light toward a water and a light receiving unit that receives light emitted from the light emitting unit and transmitted through the water, and measures the turbidity of the water based on an amount of the light received by the light receiving unit.

### [Regarding Flow Cell]

A flow cell has been used to determine the water quality of a liquid using a light source and a sensor. For example, Patent Document 2 describes a flow cell having an inflow path and an outflow path and a container body.

Patent Document 1: Japanese Unexamined Patent Application Publication No. 2015-054284

Patent Document 2: International Publication No. WO 2014/027172

### [Regarding Water Quality Determination Device and Water Quality Determination Method]

Incidentally, when determining the water quality of the water with low turbidity, such as tap water, it is necessary to use a highly accurate sensor as the light receiving unit, but such highly accurate sensors are expensive, which increase the cost. On the other hand, low-accuracy sensors are inexpensive, which reduce the cost, but they cannot accurately determine the water quality.

### SUMMARY OF THE INVENTION

The present invention has been developed in view of the above problems, and its purpose is to provide a water quality determination device and a water quality determination method capable of improving determination accuracy of water quality while reducing costs.

### [Regarding Flow Cell]

In the flow cell described in Patent Document 2, a ratio of a cross-sectional area of an inflow path to a cross-sectional area of the container body is large, and a flow velocity of a liquid changes significantly when the liquid flows from the inflow path into an inside of a container body, causing turbulence to occur inside the container body. In addition, in the flow cell described in Patent Document 2, pressure fluctuations occur between the inflow path and the inside of the container body because the cross-sectional area changes between the inflow path and the container body.

In the flow cell described in Patent Document 2, a turbulence occurs inside the container body and a pressure fluctuation occurs between the inflow path and the inside of the container body, causing air bubbles to form inside the container body. The generation of such air bubbles causes a measurement error in the sensor.

The present invention is based on the above problems, and its purpose is to provide a flow cell that can suppress or avoid the measurement failure due to the passage of air bubbles.

### [Regarding Water Quality Determination Device and Water Quality Determination Method]

A water quality determination device according to the present invention includes: a first light emitting unit and a second light emitting unit that emit lights toward a flow path; a first light receiving unit that receives the light emitted from the first light emitting unit through the flow path; and a second light receiving unit that receives the light emitted from the second light emitting unit through the flow path. The first light emitting unit and the first light receiving unit are arranged opposite each other through the flow path. The second light emitting unit and the second light receiving unit are arranged opposite each other through the flow path. An optical path length from the second light emitting unit to the second light receiving unit is longer than an optical path length from the first light emitting unit to the first light receiving unit.

The water quality determination device according to the present invention preferably includes a determination unit that determines a water quality of a water flowing through the flow path based on an amount of the light detected by the first light receiving unit and an amount of the light detected by the second light receiving unit.

In the water quality determination device according to the present invention, the determination unit may determine whether or not the amount of the light detected by the first light receiving unit and the amount of the light detected by the second light receiving unit are equal to or more than respective predetermined threshold values.

In the water quality determination device according to the present invention, the first light emitting unit and the first light receiving unit are preferably arranged along a short-side direction of the flow path, and the second light emitting unit and the second light receiving unit are preferably arranged along a long-side direction of the flow path.

Furthermore, a water quality determination method according to the present invention includes: a first detection step of receiving a light emitted from a first light emitting unit toward a flow path by a first light receiving unit through the flow path; and a second detection step of receiving a light emitted from a second light emitting unit toward the flow path by a second light receiving unit through the flow path. The first light emitting unit and the first light receiving unit are disposed opposite each other through the flow path. The second light emitting unit and the second light receiving unit are disposed opposite each other through the flow path. An optical path length from the second light emitting unit to the second light receiving unit is longer than an optical path length from the first light emitting unit to the first light receiving unit.

### [Regarding Flow Cell]

A flow cell according to the present invention includes: a container body having an inlet and an outlet for a liquid, and a flow velocity control path provided in a region between the inlet and the outlet; a light emitting unit capable of emitting a light toward the liquid flowing in the flow velocity control path; and a light receiving unit that receives the light emitted from the light emitting unit. The flow velocity control path includes: a control path inlet into which the liquid flowing in from the inlet is inflowed; and a control path outlet from which the liquid flowing into the control path inlet is outflowed. An opening area of the control path inlet and an opening area of the control path outlet are equal.

In the flow cell according to the present invention, the light emitting unit is preferably provided at one end portion in a flow path direction of the flow velocity control path, and the light receiving unit is preferably provided at the other end portion in the flow path direction of the flow velocity control path.

In the flow cell according to the present invention, a flow path cross-sectional area of the flow velocity control path may be equal to the opening area of the control path inlet and the opening area of the control path outlet.

In the flow cell according to the present invention, a flow path cross-sectional area of the flow velocity control path may be equal to the opening area of at least one of the inlet and the outlet.

In the flow cell according to the present invention, one of the light emitting unit and the light receiving unit provided on a downstream side of the flow velocity control path may be provided at a position in a height direction different from the other of the light emitting unit and the light receiving unit provided on an upstream side of the flow velocity control path.

In the flow cell according to the present invention, the container body preferably has a light-emitting unit side mounting portion capable of mounting the light emitting unit and a light-receiving unit side mounting portion capable of mounting the light receiving unit, the light-emitting unit side mounting portion preferably has a through hole that exposes the light emitting unit to an inside of the flow velocity control path, and the light-receiving unit side mounting portion preferably has a through hole that exposes the light receiving unit to the inside of the flow velocity control path.

In the flow cell according to the present invention, the container body may have a plurality of flow velocity control paths.

### [Regarding Water Quality Determination Device and Water Quality Determination Method]

With the water quality determination device and the water quality determination method of the present invention, it is possible to improve the determination accuracy of the water quality while reducing costs.

### [Regarding Flow Cell]

With the flow cell of the present invention, it is possible to suppress or avoid the measurement failure due to the passage of air bubbles.

Other systems, methods, aspects, features, embodiments and advantages of the system and method disclosed herein will be, or will become, apparent to one having ordinary skill in the art upon examination of the following drawings and detailed description. It is intended that all such additional systems, methods, aspects, features, embodiments and advantages be included within this description, and be within the scope of the accompanying claims.

### BRIEF DESCRIPTION OF THE DRAWINGS

FIG. 1 is a schematic diagram illustrating a configuration of a water quality determination device according to this embodiment.

FIG. 2 is a block diagram illustrating a configuration of a control device according to this embodiment.

FIG. 3 is a diagram illustrating an example of determination by a determination unit according to this embodiment.

FIG. 4 is a schematic diagram illustrating a flow cell according to this embodiment.

FIG. 5 is a schematic diagram illustrating a flow cell according to a modification.

FIG. 6 is a schematic diagram illustrating a flow cell according to another modification.

### DETAILED DESCRIPTION OF THE INVENTION

The present invention will now be described with reference to preferred embodiments for carrying out the invention. The following embodiments are not intended to limit the scope of the invention, and not all of the features described in the embodiments are necessarily essential to the invention. In the embodiments, the scale and dimensions of the components may be exaggerated, and some components may be omitted.

### [Overall Configuration of Water Quality Determination Device]

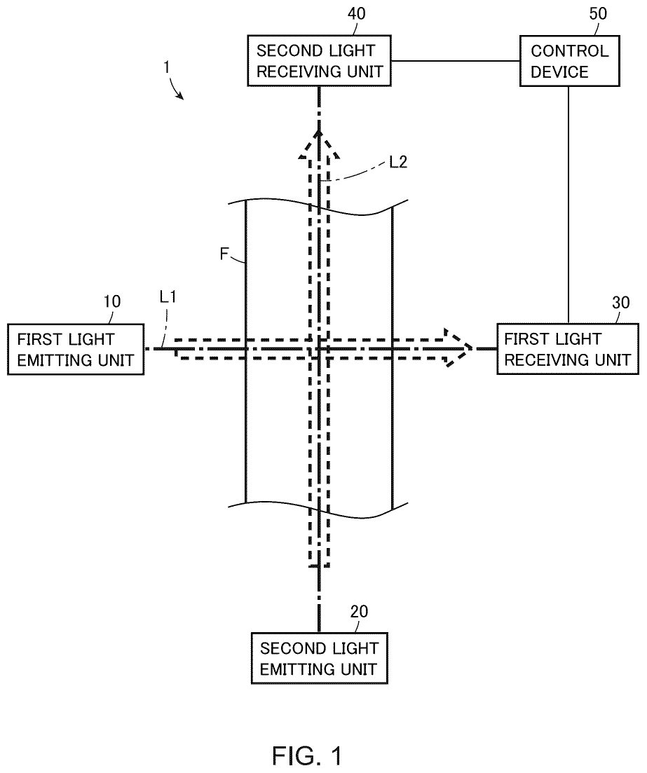

The configuration of the water quality determination device is explained with reference to FIGS. 1 to 3. As illustrated in FIG. 1, a water quality determination device 1 according to this embodiment includes a first light emitting unit 10 and a second light emitting unit 20 that emit lights toward a flow path F, a first light receiving unit 30 that receives the light emitted from the first light emitting unit 10 through the flow path F, a second light receiving unit 40 that receives the light emitted from the second light emitting unit 20 through the flow path F, and a control device 50 electrically connected to the first light receiving unit 30 and the second light receiving unit 40.

The water quality determination device 1 according to this embodiment is suitable for use in determining a water quality of a water with low turbidity, such as tap water. However, an application of the water quality determination device 1 according to this embodiment is not limited thereto, and it may also be used to determine the water quality of the water with high turbidity.

### [Configuration of First Light Emitting Unit, Second Light Emitting Unit, First Light Receiving Unit, and Second Light Receiving Unit]

The first light emitting unit 10 and the second light emitting unit 20 include, for example, any light emitting elements such as LEDs. The first light receiving unit 30 and the second light receiving unit 40 include, for example, any light receiving elements such as photodiodes. The first light receiving unit 30 is configured to receive the light emitted from the first light emitting unit 10 and transmitted through the flow path F. Similarly, the second light receiving unit 40 is configured to receive the light emitted from the second light emitting unit 20 and transmitted through the flow path F.

The light emitting element of the first light emitting unit 10 and the light emitting element of the second light emitting unit 20 may be the same or different, but it is preferable that they be the same from the viewpoint of accurately performing water quality determination. Similarly, the light receiving element of the first light receiving unit 30 and the light receiving element of the second light receiving unit 40 may be the same or different, but it is preferable that they be the same from the viewpoint of accurately performing water quality determination.

The first light emitting unit 10 and the first light receiving unit 30, and the second light emitting unit 20 and the second light receiving unit 40 are arranged such that an optical path length from the second light emitting unit 20 to the second light receiving unit 40 is longer than an optical path length from the first light emitting unit 10 to the first light receiving unit 30.

In this embodiment, the first light emitting unit 10 and the first light receiving unit 30 are disposed opposite each other along a short-side direction of the flow path F, and the second light emitting unit 20 and the second light receiving unit 40 are disposed opposite each other along a long-side direction of the flow path F. Specifically, as illustrated in FIG. 1, when viewed from a direction perpendicular to the opposite direction of the first light emitting unit 10 and the first light receiving unit 30 and perpendicular to the opposite direction of the second light emitting unit 20 and the second light receiving unit 40, the first light emitting unit 10 and the first light receiving unit 30, and the second light emitting unit 20 and the second light receiving unit 40 are arranged such that a line L1 connecting the first light emitting unit 10 and the first light receiving unit 30 and a line L2 connecting the second light emitting unit 20 and the second light receiving unit 40 intersect. That is, in this embodiment, a measurement point in the flow path F by the first light emitting unit 10 and the first light receiving unit 30 is the same as a measurement point in the flow path F by the second light emitting unit 20 and the second light receiving unit 40.

Accordingly, since the measurement point in the flow path F by the first light emitting unit 10 and the first light receiving unit 30 are the same as the measurement point in the flow path F by the second light emitting unit 20 and the second light receiving unit 40, that is, since the first light emitting unit 10 and the first light receiving unit 30 and the second light emitting unit 20 and the second light receiving unit 40 measure the same flow velocity point in the flow path F, thereby enabling accurate water quality determination.

In this embodiment, the first light emitting unit 10 and the first light receiving unit 30 are arranged along the short-side direction of the flow path F, and the second light emitting unit 20 and the second light receiving unit 40 are arranged along the long-side direction of the flow path F, but this is not limited thereto. At least one of the first light emitting unit 10 and the first light receiving unit 30, and the second light emitting unit 20 and the second light receiving unit 40 may be arranged at an inclination with respect to the flow path direction of the flow path F. In this embodiment, the first light emitting unit 10 and the first light receiving unit 30 are described as being arranged opposite each other, but this is not limited thereto. For example, the first light emitting unit 10 and the first light receiving unit 30 may be arranged in parallel, and the light emitted from the first light emitting unit 10 may be reflected by a reflection portion such as a mirror or the like and received by the first light receiving unit 30. Furthermore, similarly for the second light emitting unit 20 and the second light receiving unit 40, the second light emitting unit 20 and the second light receiving unit 40 may also be arranged in parallel, and the light emitted from the second light emitting unit 20 may be reflected by a reflection portion and received by the second light receiving unit 40. In addition, depending on a presence or absence of the reflection portion, the number of installations, and the like, the optical path length from the first light emitting unit 10 to the first light receiving unit 30 may be different from the optical path length from the second light emitting unit 20 to the second light receiving unit 40.

### [Configuration of Control Device]

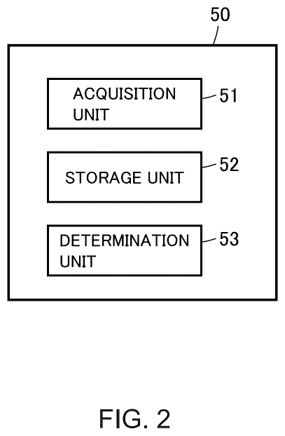

As illustrated in FIG. 2, the control device 50 includes an acquisition unit 51 that acquires light amount information detected by the first light receiving unit 30 and the second light receiving unit 40 from the first light receiving unit 30 and the second light receiving unit 40, a storage unit 52 that stores threshold information, and a determination unit 53 that determines the water quality of the water flowing through the flow path F based on the light amount detected by the first light receiving unit 30 and the light amount detected by the second light receiving unit 40.

Here, “light amount” may be expressed, for example, as light intensity or a measured value. In addition, “threshold information” is reference value information for determining whether the water flowing through the flow path F is of good quality, and may be set arbitrarily.

The determination unit 53 is configured to determine whether or not the light amount detected by the first light receiving unit 30 and the light amount detected by the second light receiving unit 40 are equal to or more than respective predetermined threshold values, based on the light amount information detected by the first light receiving unit 30 and the second light receiving unit 40 acquired by the acquisition unit 51, and the threshold information stored in the storage unit 52.

Furthermore, the determination unit 53 may be configured to determine whether or not the water flowing through the flow path F is of good quality and to determine whether or not a water purification treatment apparatus (not illustrated) that performs a water purification treatment of the water flowing through the flow path F is normal or not, based on the determination result of whether or not the amount of light detected by the first light receiving unit 30 and the amount of light detected by the second light receiving unit 40 are equal to or more than the respective predetermined threshold values.

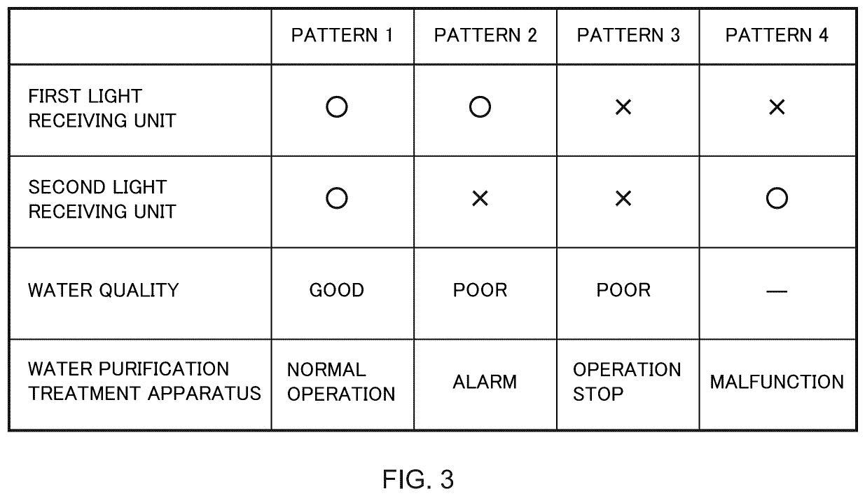

Specifically, as illustrated in FIG. 3, the determination unit 53 determines that the water quality of the water flowing through the flow path F is good when the amount of light detected by the first light receiving unit 30 and the amount of light detected by the second light receiving unit 40 are both equal to or more than the respective predetermined threshold values and determines that the water quality of the water flowing through the flow path F is poor when at least one of the amount of light detected by the first light receiving unit 30 and the amount of light detected by the second light receiving unit 40 is less than the predetermined threshold value.

Furthermore, as illustrated in FIG. 3, the determination unit 53 may be configured such that when the amount of light detected by the first light receiving unit 30 and the amount of light detected by the second light receiving unit 40 are both equal to or more than the respective predetermined threshold values, the determination unit 53 determines that the water purification treatment apparatus is operating normally, when only the amount of light detected by the first light receiving unit 30 is less than the predetermined threshold value, the determination unit 53 determines to issue an alarm to the water purification treatment apparatus, when the amount of light detected by the first light receiving unit 30 and the amount of light detected by the second light receiving unit 40 are less than the respective predetermined thresholds, the determination unit 53 determines that the water purification treatment apparatus should be stopped, and when only the amount of light detected by the second light receiving unit 40 is less than the predetermined threshold, the determination unit 53 determines that at least one of the first light receiving unit 30, the second light receiving unit 40, and the water purification treatment apparatus is malfunctioning.

### [Water Quality Determination Method]

Next, a water quality determination method using the water quality determination device 1 according to this embodiment will be described.

[first Detection Step and Second Detection Step]

First, in the first detection step, the light emitted from the first light emitting unit 10 toward the flow path F is received by the first light receiving unit 30 through the flow path F. Next, in the second detection step, the light emitted from the second light emitting unit 20 toward the flow path F is received by the second light receiving unit 40 through the flow path F.

### [Acquisition Step and Determination Step]

Thereafter, in the acquisition step, the acquisition unit 51 acquires the light amount information detected by the first light receiving unit 30 and the second light receiving unit 40 from the first light receiving unit 30 and the second light receiving unit 40. Finally, in the determination step, the determination unit 53 determines whether or not the light amount detected by the first light receiving unit 30 and the light amount detected by the second light receiving unit 40 are equal to or more than the respective predetermined threshold values, based on the light amount information detected by the first light receiving unit 30 and the second light receiving unit 40 acquired by the acquisition unit 51, and the threshold information stored in the storage unit 52.

[Advantages of Water Quality Determination Device according to Embodiment]

Accordingly, the water quality determination device 1 according to this embodiment includes the first light emitting unit 10 and the second light emitting unit 20 that emit the light toward the flow path F, the first light receiving unit 30 that receives the light emitted from the first light emitting unit 10 through the flow path F, the second light receiving unit 40 that receives the light emitted from the second light emitting unit 20 through the flow path F. The optical path length from the second light emitting unit 20 to the second light receiving unit 40 is longer than the optical path length from the first light emitting unit 10 to the first light receiving unit 30.

According to the water quality determination device 1 having such a configuration, since the optical path length from the first light emitting unit 10 to the first light receiving unit 30 is different from the optical path length from the second light emitting unit 20 to the second light receiving unit 40, even when a sensor with low accuracy is used, the slight difference in the light amount information detected by the first light receiving unit 30 and the second light receiving unit 40 allows detecting a slight difference in turbidity. The water quality can be accurately determined even for a water with a low degree of contamination. Therefore, there is an advantage in that the determination accuracy of water quality can be improved while reducing cost.

In addition, the water quality determination device 1 according to this embodiment includes the determination unit 53 that determines the water quality of the water flowing through the flow path F based on the amount of the light detected by the first light receiving unit 30 and the amount of the light detected by the second light receiving unit 40. With the water quality determination device 1 having such a configuration, there is no need to measure turbidity, or the like, and the water quality can be determined simply by measuring the amount of light, thereby improving the efficiency of the water quality determination.

Furthermore, in the water quality determination device 1 according to this embodiment, the determination unit 53 determines whether or not the amount of the light detected by the first light receiving unit 30 and the amount of the light detected by the second light receiving unit 40 are equal to or more than the respective predetermined threshold values. According to the water quality determination device 1 having such a configuration, there is an advantage in that the determination accuracy of the water quality can be improved and rapid determination can be enabled by comparing the measured values detected by the first light receiving unit 30 and the second light receiving unit 40 with the predetermined threshold values.

In the water quality determination device 1 according to this embodiment, the first light emitting unit 10 and the first light receiving unit 30 are arranged along the short-side direction of the flow path F, and the second light emitting unit 20 and the second light receiving unit 40 are arranged along the long-side direction of the flow path F. According to the water quality determination device 1 having such a configuration, there is an advantage that the arrangement of the first light emitting unit 10 and the first light receiving unit 30, and the second light emitting unit 20 and the second light receiving unit 40 is simple.

### [Modification of Water Quality Determination Device]

The water quality determination device according to the present invention is not limited to the above-described embodiment, and various modifications may be made within the scope of the technical concept of the present invention.

For example, in the above embodiment, the determination unit 53 is described as performing the determination using the threshold information stored in the storage unit 52, but this is not limited thereto, and for example, it may be configured to perform AI determination. In addition, the determination unit 53 may calculate the turbidity and color of the water and determine the water quality based on the calculated turbidity and color of the water.

In the above-described embodiment, the first light receiving unit 30 and the second light receiving unit 40 are described as directly receiving the light transmitted through the flow path F, but this is not limited thereto, and for example, the device may further include a reflection portion for reflecting the light transmitted through the flow path F, and the first light receiving unit 30 and the second light receiving unit 40 may be configured to receive the light reflected by the reflection portion. In this case, the first light receiving unit 30 and the second light receiving unit 40 may be disposed at either position.

Furthermore, in the above-described embodiment, the measurement point in the flow path F by the first light emitting unit 10 and the first light receiving unit 30 and the measurement point in the flow path F by the second light emitting unit 20 and the second light receiving unit 40 are described as being the same, but this is not limited thereto, and the measurement point in the flow path F by the first light emitting unit 10 and the first light receiving unit 30 and the measurement point in the flow path F by the second light emitting unit 20 and the second light receiving unit 40 may be different.

In the above-described embodiment, the control device 50 is described as including the acquisition unit 51, the storage unit 52, and the determination unit 53, but in addition to these, the control device 50 may also include a display unit capable of displaying the determination result by the determination unit 53.

Furthermore, in the above-described embodiment, the water quality determination device 1 is described as including the first light emitting unit 10 and the first light receiving unit 30, and the second light emitting unit 20 and the second light receiving unit 40, but the number of the light emitting units and the light receiving units is not limited. That is, the water quality determination device 1 may include a third light emitting unit and a third light receiving unit, or may include four or more sets of light emitting units and light receiving units. In particular, when three or more sets of light emitting units and light receiving units are provided, it is possible to determine the water quality level, and the like in a more detailed and segmented manner than in the above-described embodiment including the two sets of the light emitting units 10, 20 and the light receiving units 30, 40.

It is clear from the description of the claims that the above-described modifications are included in the scope of the present invention.

### [Overall Configuration of Flow Cell]

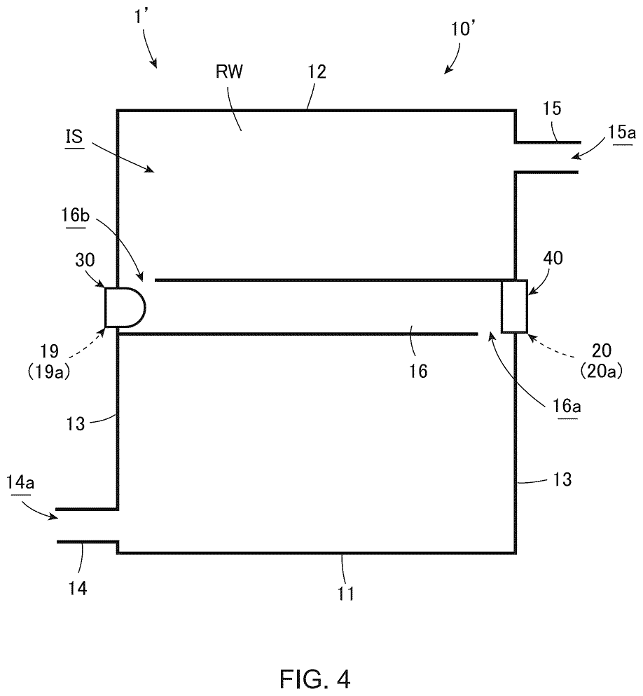

The configuration of the flow cell is described with reference to FIGS. 4 to 6. As illustrated in FIG. 4, a flow cell 1′ according to this embodiment includes a container body 10′ and a light emitting unit 30 and a light receiving unit 40 provided in the container body 10′.

### [Structure of Container Body]

As illustrated in FIG. 4, the container body 10′ includes a bottom plate portion 11 and a top plate portion 12 formed in a long rectangular shape, a pair of side wall portions 13 formed from both ends in a long-side direction of the bottom plate portion 11 to both ends in the long-side direction of the top plate portion 12, a front wall portion and a rear wall portion RW, which are formed from both ends in a short-side direction of the bottom plate portion 11 to both ends in the short-side direction of the top plate portion 12, and is formed as a box having an internal space IS as a whole. FIG. 4 is a schematic diagram illustrating the front wall portion in a transparent state.

In this specification, a direction from the bottom plate portion 11 to the top plate portion 12 (or a direction from the top plate portion 12 to the bottom plate portion 11) is defined as the “height direction.” In addition, in this specification, the direction in which the bottom plate portion 11 is located in the height direction is defined as “downward,” and the direction in which the top plate portion 12 is located is defined as “upward.”

The container body 10′ has a liquid inflow path 14 at a lower end of one side wall portion 13. The inflow path 14 is formed in a cylindrical shape having an internal space capable of allowing the liquid to flow, and is formed extending outward from an outer surface of one side wall portion 13. In addition, a distal end and a base end of the inflow path 14 are open. In this embodiment, the distal end of the inflow path 14 functions as an inlet 14a. In other words, the inlet 14a is an opening formed at the distal end of the inflow path 14.

Similarly, the container body 10′ has an outflow path 15 at an upper end portion of the other side wall portion 13. The outflow path 15 is formed in a cylindrical shape having an internal space capable of allowing the liquid to flow, and is formed extending outwardly from an outer surface of the other side wall portion 13. In addition, a distal end and a base end of the outflow path 15 are open. In this embodiment, the distal end of the outflow path 15 functions as an outlet 15a. In other words, the outlet 15a is an opening formed at the distal end of the outflow path 15.

In addition, the container body 10′ has a flow velocity control path 16 provided in a region between the inlet 14a and the outlet 15a. Specifically, the flow velocity control path 16 is provided in the internal space IS defined by the bottom plate portion 11, the top plate portion 12, the side wall portions 13, the front wall portion, and the rear wall portion RW.

The flow velocity control path 16 is formed in a cylindrical shape having an internal space capable of flowing the liquid, and is formed extending along the same direction as the opening direction (that is, a left-right direction) of the inlet 14a and the outlet 15a. Specifically, the flow velocity control path 16 is formed extending from one side wall portion 13 toward the other side wall portion 13.

In this embodiment, a flow path cross-sectional area of the flow velocity control path 16 is constant in the flow path direction of the flow velocity control path 16. In addition, the flow path cross-sectional area of the flow velocity control path 16 is preferably formed to be equal to the opening area of at least one of the inlet 14a and the outlet 15a. In this embodiment, the flow path cross-sectional area of the flow velocity control path 16 is formed to be equal to the opening areas of both the inlet 14a and the outlet 15a.

In this specification, “equal” means that the area is within ±5% of a specified area.

The flow velocity control path 16 also has a control path inlet 16a into which the liquid flowing in from the inlet 14a flows, and a control path outlet 16b from which the liquid flowing into the control path inlet 16a flows out. The control path inlet 16a is an opening formed at a lower end of the flow velocity control path 16 at one end portion of the flow path direction of the flow velocity control path 16. On the other hand, the control path outlet 16b is an opening formed at an upper end of the flow velocity control path 16 at the other end portion of the flow path direction of the flow velocity control path 16.

Here, an opening area of the control path inlet 16a and an opening area of the control path outlet 16b are formed to be equal. In addition, the flow path cross-sectional area of the flow velocity control path 16 is formed to be equal to the opening area of the control path inlet 16a and the opening area of the control path outlet 16b.

The container body 10′ also includes a light-emitting unit side mounting portion 19 capable of mounting the light emitting unit 30 and a light-receiving unit side mounting portion 20 capable of mounting the light receiving unit 40. The light-emitting unit side mounting portion 19 is provided on one of the side wall portions 13 and has a through hole 19a for exposing the light emitting unit 30 to an inside of the flow velocity control path 16. Similarly, the light-receiving unit side mounting portion 20 is provided on the other side wall portion 13 and has a through hole 20a for exposing the light receiving unit 40 to the inside of the flow velocity control path 16. That is, the light-emitting unit side mounting portion 19 and the light-receiving unit side mounting portion 20 are each connected to the flow velocity control path 16.

The container body 10′ may be made of, for example, a shielding metal or a resin plastic. That is, as will be described later, in the flow cell 1′ according to this embodiment, since the light emitting unit 30 is provided at one end portion in the flow path direction of the flow velocity control path 16, and the light receiving unit 40 is provided at the other end portion in the flow path direction of the flow velocity control path 16, unlike the conventional flow cell in which the light emitting unit and the light receiving unit are respectively mounted to the outer surface of the container body, a non-light-transmissive material can be used. In addition, the container body 10′ is preferably formed by integral molding using the above-described materials. The molding material and molding method of the container body 10′ are not limited thereto, and for example, a light-shielding material may be used to improve the measurement accuracy of the light receiving unit 40, a material that is easy to mold or process may be used, or other various known molding materials and molding methods may be used.

### [Structure of Light Emitting Unit and Light Receiving Unit]

As illustrated in FIG. 4, the light emitting unit 30 is provided at one end portion in the flow path direction of the flow velocity control path 16. Specifically, the light emitting unit 30 is configured to be mounted on the light-emitting unit side mounting portion 19 of the container body 10′. In addition, the light emitting unit 30 is configured so as to seal one end portion of the flow velocity control path 16 in the flow path direction when mounted to the light-emitting unit side mounting portion 19.

The light emitting unit 30 having the above configuration is, for example, provided with an arbitrary light emitting element such as an LED, and is configured to irradiate light toward the liquid flowing through the flow velocity control path 16.

As illustrated in FIG. 4, the light receiving unit 40 is provided at the other end portion in the flow path direction of the flow velocity control path 16. Specifically, the light receiving unit 40 is configured to be mounted on the light-receiving unit side mounting portion 20 of the container body 10′. In addition, the light receiving unit 40 is configured to seal the other end portion of the flow velocity control path 16 in the flow path direction when mounted on the light-receiving unit side mounting portion 20.

The light receiving unit having the above configuration includes, for example, an arbitrary light receiving element such as a photodiode, and is configured to receive the light emitted from the light emitting unit 30. Specifically, the light receiving unit 40 is configured to receive the light emitted from the light emitting unit 30 and transmitted through the liquid flowing in the flow velocity control path 16.

### [use Method of Flow Cell]

The flow cell 1′ according to this embodiment is used by connecting the inflow path 14 to the other flow path on the upstream side and connecting the outflow path 15 to the other flow path on the downstream side. The liquid flowing into the inflow path 14 from the other flow path on the upstream side flows through the internal space IS, the flow velocity control path 16, and the outflow path 15, and then flows into the other flow path on the downstream side.

In addition, while the liquid is flowing through the flow velocity control path 16, the light emitting unit 30 emits a light toward the liquid flowing through the flow velocity control path 16, and the light receiving unit 40 receives the light that has passed through the liquid flowing through the flow velocity control path 16. The light quantity value (measured value) received by the light receiving unit 40 is sent to a control device (not illustrated) and the water quality, such as the turbidity and color of the liquid flowing through the flow cell 1′, is determined by the control device.

[Advantages of Flow Cell according to Embodiment]

Accordingly, the flow cell 1′ according to this embodiment includes the container body 10′ including the liquid inlet 14a and the outlet 15a, and the flow velocity control path 16 provided in the region between the inlet 14a and the outlet 15a, the light emitting unit 30 capable of emitting a light toward the liquid flowing through the flow velocity control path 16, and the light receiving unit 40 capable of receiving the light emitted from the light emitting unit 30. The flow velocity control path 16 has the control path inlet 16a into which the liquid flowing in from the inlet 14a flows, and the control path outlet 16b from which the liquid flowing into the control path inlet 16a flows out. The opening area of the control path inlet 16a and the opening area of the control path outlet 16b are equal.

According to the flow cell 1′ having such a configuration, since the opening area of the control path inlet 16a and the opening area of the control path outlet 16b are equal, the liquid flow velocity in the flow velocity control path 16 does not change. This has the advantage of preventing the occurrence of turbulent flow and the pressure fluctuation in the flow velocity control path 16, thereby suppressing or avoiding the measurement failure due to the generation or passage of air bubbles. The flow cell 1′ according to this embodiment is also suitable for measuring a liquid with relatively high flow velocity (liquids with flow velocity faster than 100 mL/min to 250 mL/min).

Furthermore, according to the flow cell 1′ having the above configuration, since the container body 10′ includes a flow velocity control path 16, there is no need to provide separate piping for measurement, and a continuous measurement is also possible. That is, in the conventional flow cell, after the liquid is inflowed into and accumulated in the container body, the measurement is performed by a light receiving unit (sensor) while a flow velocity of the liquid is stabilized, but in the flow cell 1′ according to this embodiment, the liquid accumulation step and the flow velocity stabilization step can be omitted, and the continuous measurement can be performed.

In the flow cell 1′ according to this embodiment, the light emitting unit 30 is provided at one end portion in the flow path direction of the flow velocity control path 16, and the light receiving unit 40 is provided at the other end portion in the flow path direction of the flow velocity control path 16. According to the flow cell 1′ having such a configuration, the light emitting unit 30 and the light receiving unit 40 are provided in the flow velocity control path 16, and the light is directly emitted and received with respect to the liquid flowing in the flow velocity control path 16 (that is, the light is not emitted and received through the side wall portion 13 or the like). Thus, it is possible to measure the liquid with low turbidity or low chromaticity and distinguish the slight differences in turbidity or chromaticity. This has an advantage of greatly improving the measurement accuracy compared with the conventional flow cell in which the light emitting unit and the light receiving unit are each mounted on the outer surface of the container body.

In the flow cell 1′ according to this embodiment, the flow path cross-sectional area of the flow velocity control path 16 is equal to the opening area of the control path inlet 16a and the opening area of the control path outlet 16b. According to the flow cell 1′ having such a configuration, there is an advantage that the measurement failure due to the generation of bubbles or the passage of bubbles in the flow velocity control path 16 can be suppressed or avoided.

In the flow cell 1′ according to this embodiment, the flow path cross-sectional area of the flow velocity control path 16 is equal to the opening area of at least one of the inlet 14a and the outlet 15a. According to the flow cell 1′ having such a configuration, there is an advantage that the generation and retention of air bubbles in the flow velocity control path 16 can be further suppressed.

In the flow cell 1′ according to this embodiment, the container body 10′ includes the light-emitting unit side mounting portion 19 capable of mounting the light emitting unit 30 and a light-receiving unit side mounting portion 20 capable of mounting the light receiving unit 40. The light-emitting unit side mounting portion 19 has the through hole 19a that exposes the light emitting unit 30 to the inside of the flow velocity control path 16. The light-receiving unit side mounting portion 20 has the through hole 20a that exposes the light receiving unit 40 to the inside of the flow velocity control path 16. According to the flow cell 1′ having such a configuration, since the light emitting unit 30 and the light receiving unit 40 are exposed to the inside of the flow velocity control path 16, it is possible, for example, to measure the liquid having low turbidity or low chromaticity, and to distinguish slight differences in turbidity or chromaticity. There is an advantage that the measurement accuracy is greatly improved compared with the conventional flow cell in which the light emitting unit and the light receiving unit are mounted on the outer surface of the container body. In addition, since the light emitting unit 30 and the light receiving unit 40 are detachable from the container body 10′, there is also the advantage that they can be easily replaced in the event of failure of the light emitting unit 30 and the light receiving unit 40.

### [Modification of Flow Cell]

The flow cell according to the present invention is not limited to the above-described embodiment, and various modifications may be made within the scope of the technical concept of the present invention.

For example, in the above embodiment, the light emitting unit 30 is provided at one end portion of the flow path direction of the flow velocity control path 16, and the light receiving unit 40 is provided at the other end portion of the flow path direction of the flow velocity control path 16, but these are not limited thereto and may be provided at any position.

In the above embodiment, it has been described that only one light emitting unit 30 is provided, but this is not limited thereto, and two or more light emitting units 30 may be provided. In this case, one light emitting unit 30 (first light emitting unit) may be provided at one end portion in the flow path direction of the flow velocity control path 16, and the other light emitting unit 30 (second light emitting unit) may be provided on an inner surface perpendicular to the flow path direction of the flow velocity control path 16. By providing two or more light emitting units 30 (first light emitting unit and second light emitting unit), it is possible to identify not only the water quality, such as the turbidity and chromaticity of the liquid flowing through the flow velocity control path 16, but also slight differences and changes in low turbidity and low chromaticity.

Furthermore, in the above embodiment, the flow path cross-sectional area of the flow velocity control path 16 is described as being equal to the opening area of the control path inlet 16a and the opening area of the control path outlet 16b, but this is not limited thereto, and a configuration in which they are not equal is also possible.

In the above-described embodiment, the flow path cross-sectional area of the flow velocity control path 16 is described as being equal to the opening area of at least one of the inlet 14a and the outlet 15a, but this is not limited thereto, and a configuration in which they are not equal is also acceptable.

Furthermore, in the above-described embodiment, the light emitting unit 30 and the light receiving unit 40 are described as being exposed to the inside of the flow velocity control path, but this is not limited thereto, and the light emitting unit 30 and the light receiving unit 40 may also be configured so as not to be exposed to the inside of the flow velocity control path 16. That is, the light emitting unit 30 and the light receiving unit 40 may also be configured so as to be provided on the outer surface of the side wall portion 13.

In addition, for example, the light emitting unit 30 and the light receiving unit 40 may each constitute a part of the side wall portion 13.

Furthermore, in the above-described embodiment, the flow velocity control path 16 is described as being formed in the cylindrical shape having the internal space capable of flowing the liquid, but this is not limited thereto. For example, the flow velocity control path 16 may have two plate-like partition portions extending from the front wall portion to the rear wall portion RW of the container body 10′, and the two partition portions may be arranged at different heights.

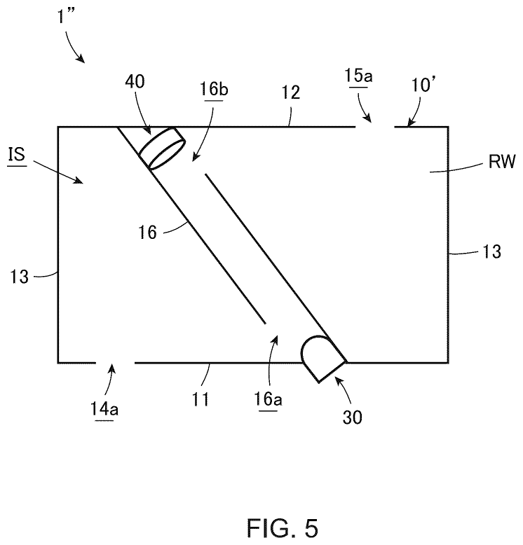

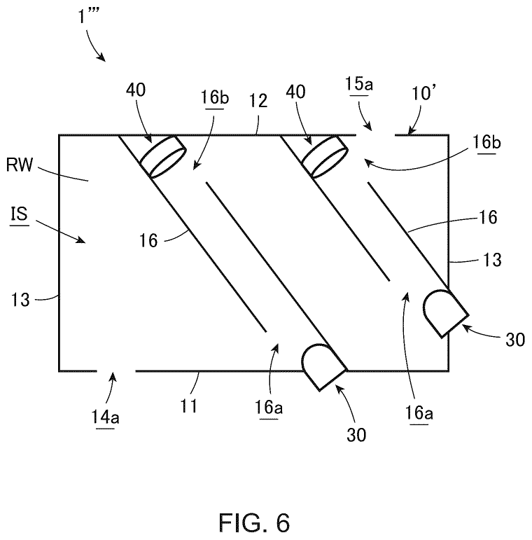

In the above-described embodiment, the container body 10′ has the inflow path 14 and the outflow path 15, but this is not limited thereto. For example, as illustrated in a flow cell 1″ in FIG. 5 and a flow cell 1′″ in FIG. 6, the container body 10′ does not have the inflow path 14 or the outflow path 15, and the inlet 14a is formed directly in the bottom plate portion 11, and the outlet 15a may be formed directly in the top plate portion 12. The flow cell 1″ illustrated in FIG. 5 and the flow cell 1′″ illustrated in FIG. 6 may have the inflow path 14 and the outflow path 15 as in the flow cell 1′ illustrated in FIG. 4.

Below, the configuration of flow cell 1″ illustrated in FIG. 5 and flow cell 1′″ illustrated in FIG. 6 will be described, focusing on the differences from the configuration of flow cell 1′ illustrated in FIG. 4.

The flow cell 1″ illustrated in FIG. 5 and the flow cell 1′″ illustrated in FIG. 6 differ from the flow cell 1′ illustrated in FIG. 4 in that the inlet 14a is formed at one end portion (the left end portion in FIGS. 5 and 6) in the long-side direction of the bottom plate portion 11, the outlet 15a is formed at the end of the top plate portion 12 in the long-side direction opposite to the side where the inlet 14a is located (the right end in FIG. 5 and FIG. 6), and the flow velocity control path 16 is formed extending from the bottom plate portion 11 toward the top plate portion 12. With this configuration, before the liquid flows into the control path inlet 16a of the flow velocity control path 16, bubbles generated when the liquid flows from the inlet 14a into the internal space IS of the container body 10′ and solid matter contained in the liquid are removed (that is, bubbles and solid matter rise in the internal space IS of the container body 10′ and move toward the top plate portion 12, and are discharged from the flow cell 1″ (or flow cell 1′″) through the communicating outlet 15a), the measurement failures due to the generation or passage of the air bubbles in the flow velocity control path 16 are suppressed or avoided, and the measurement errors in the light receiving unit 40 (sensor) are reduced. In the flow cell 1″ illustrated in FIG. 5 and the flow cell 1′″ illustrated in FIG. 6, the flow velocity control path 16 is inclined in the height direction, but it may also be provided along the height direction.

In the flow cell 1″ illustrated in FIG. 5 and the flow cell 1′″ illustrated in FIG. 6, the air bubbles removed before the liquid flows into the control path inlet 16a of the flow velocity control path 16 are discharged from the outlet 15a. In addition, the container body 10′ may have a degassing hole capable of discharging the air bubbles, and the discharge may be performed in combination with the discharge by the outlet 15a.

The flow cell 1″ illustrated in FIG. 5 and the flow cell 1′″ illustrated in FIG. 6 differ from the flow cell 1′ illustrated in FIG. 4 in that one of the light emitting unit 30 and the light receiving unit 40 provided on the downstream side of the flow velocity control path 16 is provided at a position different from the other of the light emitting unit 30 and the light receiving unit 40 provided on the upstream side of the flow velocity control path 16 in the height direction. In the flow cell 1″ illustrated in FIG. 5 and the flow cell 1′″ illustrated in FIG. 6, the light receiving unit 40 is provided at a position higher than the light emitting unit 30. With this configuration, since the flow velocity control path 16 is formed from the bottom plate portion 11 toward the top plate portion 12, the bubbles and solids can be removed before liquid flows into the control path inlet 16a of the flow velocity control path 16 as described above.

The flow cell 1′″ illustrated in FIG. 6 differs from the flow cell 1′ illustrated in FIG. 4 in that it has a plurality (the two in the example illustrated in FIG. 6) of the flow velocity control path 16, the light emitting unit 30, and the light receiving unit 40. With this configuration, it is possible to more accurately identify the water quality, such as the turbidity and chromaticity of the liquid flowing through the flow velocity control path 16, as well as the slight differences in the low turbidity and the low chromaticity and changes therein, by utilizing differences in the measured values due to differences in the optical path length and the light source. In the flow cell 1′″ illustrated in FIG. 6, the flow path lengths of the flow velocity control paths 16 may be different or the same.

It is clear from the description of the claims that the above-described modified examples are included in the scope of the present invention.

In the foregoing description, reference is made to the accompanying drawings that form a part thereof, and in which is shown by way of illustration specific exemplary embodiments in which the disclosure may be practiced. These embodiments are described in sufficient detail to enable those skilled in the art to practice the concepts disclosed herein, and it is to be understood that modifications to the various disclosed embodiments may be made, and other embodiments may be utilized, without departing from the scope of the present disclosure. The foregoing detailed description is, therefore, not to be taken in a limiting sense.

Reference throughout this specification to “one embodiment,” “an embodiment,” “one example,” or “an example” means that a particular feature, structure, or characteristic described in connection with the embodiment or example is included in at least one embodiment of the present disclosure. Thus, appearances of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims. It is also understood that other embodiments of this invention may be practiced in the absence of an element/step not specifically disclosed herein.

What is claimed is:

1. A water quality determination device comprising:
a first light emitting unit and a second light emitting unit that emit lights toward a flow path;
a first light receiving unit that receives the light emitted from the first light emitting unit through the flow path; and
a second light receiving unit that receives the light emitted from the second light emitting unit through the flow path, wherein
the first light emitting unit and the first light receiving unit are arranged opposite each other through the flow path,
the second light emitting unit and the second light receiving unit are arranged opposite each other through the flow path, and
an optical path length from the second light emitting unit to the second light receiving unit is longer than an optical path length from the first light emitting unit to the first light receiving unit.

2. The water quality determination device according to claim 1, comprising
a determination unit that determines a water quality of a water flowing through the flow path based on an amount of the light detected by the first light receiving unit and an amount of the light detected by the second light receiving unit.

3. The water quality determination device according to claim 2, wherein
the determination unit determines whether or not the amount of the light detected by the first light receiving unit and the amount of the light detected by the second light receiving unit are equal to or more than respective predetermined threshold values.

4. The Water quality determination device according to claim 1, wherein
the first light emitting unit and the first light receiving unit are arranged along a short-side direction of the flow path, and
the second light emitting unit and the second light receiving unit are arranged along a long-side direction of the flow path.

5. A water quality determination method comprising:
a first detection step of receiving a light emitted from a first light emitting unit toward a flow path by a first light receiving unit through the flow path; and
a second detection step of receiving a light emitted from a second light emitting unit toward the flow path by a second light receiving unit through the flow path, wherein
the first light emitting unit and the first light receiving unit are disposed opposite each other through the flow path,
the second light emitting unit and the second light receiving unit are disposed opposite each other through the flow path,
an optical path length from the second light emitting unit to the second light receiving unit is longer than an optical path length from the first light emitting unit to the first light receiving unit.

6. A flow cell comprising:
a container body having an inlet and an outlet for a liquid, and a flow velocity control path provided in a region between the inlet and the outlet;
a light emitting unit capable of emitting a light toward the liquid flowing in the flow velocity control path; and
a light receiving unit that receives the light emitted from the light emitting unit, wherein
the flow velocity control path includes:
a control path inlet into which the liquid flowing in from the inlet is inflowed; and
a control path outlet from which the liquid flowing into the control path inlet is outflowed, wherein
an opening area of the control path inlet and an opening area of the control path outlet are equal.

7. The flow cell according to claim 6, wherein
the light emitting unit is provided at one end portion in a flow path direction of the flow velocity control path, and
the light receiving unit is provided at the other end portion in the flow path direction of the flow velocity control path.

8. The flow cell according to claim 6, wherein
a flow path cross-sectional area of the flow velocity control path is equal to the opening area of the control path inlet and the opening area of the control path outlet.

9. The flow cell according to claim 6, wherein
a flow path cross-sectional area of the flow velocity control path is equal to the opening area of at least one of the inlet and the outlet.

10. The flow cell according to claim 7, wherein
one of the light emitting unit and the light receiving unit provided on a downstream side of the flow velocity control path is provided at a position in a height direction different from the other of the light emitting unit and the light receiving unit provided on an upstream side of the flow velocity control path.

11. The flow cell according to claim 6, wherein
the container body has a light-emitting unit side mounting portion capable of mounting the light emitting unit and a light-receiving unit side mounting portion capable of mounting the light receiving unit,
the light-emitting unit side mounting portion has a through hole that exposes the light emitting unit to an inside of the flow velocity control path, and
the light-receiving unit side mounting portion has a through hole that exposes the light receiving unit to the inside of the flow velocity control path.

12. The flow cell according to claim 6, wherein
the container body has a plurality of flow velocity control paths.
